(19) United States
(12) Patent Application Publication
(10) Pub. No.: US 2021/0275716 A1
(43) Pub. Date: Sep. 9, 2021

(54) BRAIDED SURGICAL IMPLANTS

(71) Applicant: Embody, Inc., Norfolk, VA (US)

(72) Inventors: Michael P. Francis, Norfolk, VA (US); Nicholas Thayer, Morrisville, NC (US); Nardos Sori, Norfolk, VA (US)

(21) Appl. No.: 17/162,568

(22) Filed: Jan. 29, 2021

Related U.S. Application Data
(60) Provisional application No. 62/968,873, filed on Jan. 31, 2020.

(51) Int. Cl. A61L 17/14; A61B 17/06; D04C 1/02; D04C 1/06

(52) U.S. Cl. CPC A61L 17/14; A61B 17/06166; A61F 2/0063; D04C 1/06; D04C 1/02

(57) ABSTRACT

A strand that can be used to support repair of a soft tissue injury is disclosed. The strand comprises high strength collagen fibers and high strength biocompatible fibers, such as polyethylene fibers arranged into a strand that can be used as part of a suture or other scaffold for the repair of joints and soft tissues, such as ligaments and tendons. The fibers may be over-braided around a central core, which is itself comprised of two or more fibers. The high strength collagen fibers are strong enough to withstand the stresses imposed by industrial braiding machines and processes.

### CROSS-REFERENCE TO RELATED APPLICATIONS

This application claims the benefit of Provisional Patent Application No. 62/968,873, filed on Jan. 31, 2020, and titled “Braided and Bundled Surgical Devices and Implants,” the entire disclosure of which is hereby incorporated by reference.

### BACKGROUND

Surgical repair of injuries to various joints, ligaments and tendons is a common procedure, including those of the ankle, knee, shoulder, Achilles tendon, patellar tendon, and supraspinatus tendon, among others.

For example, in the United States, there are approximately 500,000 knee ligament ruptures annually, of which an estimated 100,000 are augmented with a scaffold implant or suture (typically a prosthetic polymer, autograft or allograft).

Collagen tape repairs are intended to provide additional mechanical stabilization post-operatively and serve as a stimulus for healing and regeneration. However, in spite of their widespread use, currently marketed scaffolds do not share the same mechanical properties of human ligaments, nor have they been shown clinically to augment cellular/tissue healing in a meaningful way.

The current standard of care for a ruptured anterior cruciate ligament (ACL) is patient autografting, where tissue is harvested (for example, from hamstring or patellar tendon) for use in place of the ruptured or torn ACL. Autograft, as well as allograft tissues, are sometimes reinforced with a permanent synthetic suture—a procedure known as a ligament “internal brace.” Allografting, where tissue is harvested from cadaveric human tendons, is also used in ACL reconstruction. ACL reconstruction with autografts or allografts entails drilling through and destroying the native ACL, eliminating its associated bone bed, nerve and blood supply, thereby killing the native cell types present within and adjoining the ACL tissue. Allografts are supply-limited, promote scar formation, may provoke an immune response, and have poorly defined turnover rates, all of which can inhibit healing.

Such products must function in a variety of challenging biomechanical environments in which multiple functional parameters must be addressed. These parameters include, for example, compatibility with bodily tissue and fluids, strength, flexibility, and biodegradability.

There is a need in the art for a system and method that addresses the shortcomings of the prior art discussed above.

### SUMMARY

In one aspect, an implantable biopolymer scaffold includes at least one braided strand, where the at least one braided strand consists essentially of high strength collagen fibers and high strength biocompatible fibers.

In another aspect, an implantable biopolymer scaffold includes a set of high strength collagen fibers braided with a set of high strength polyethylene fibers. A high strength collagen fiber of the set of high strength collagen fibers has a first ultimate tensile strength, and a high strength polyethylene fiber of the set of high strength polyethylene fibers has a second ultimate tensile strength. The first ultimate tensile strength is at least about 1 percent, 3 percent, 5 percent or 10 percent of the second ultimate tensile strength.

In another aspect, a braided strand includes a set of high strength collagen fibers braided with a set of high strength polyethylene fibers. A high strength collagen fiber of the set of high strength collagen fibers has a first ultimate tensile strength, and a high strength polyethylene fiber of the set of high strength polyethylene fibers has a second ultimate tensile strength. The first ultimate tensile strength is at least about 1 percent, 3 percent, 5 percent or 10 percent of the second ultimate tensile strength.

In yet other embodiments, the invention relates to methods of repairing an injured joint, ligament or tendon, involving the implanting of an implantable biopolymer scaffold according to the invention. In some procedures, the scaffold has a form factor of a brace. Related procedures include the securing of such an implant by suturing the implant into a desired position with a suture comprised of fibers according to the invention. Such methods may include fixation using various anchors as would be known to persons skilled in the art. Other procedures involve the closing of an incision or wound or repairing injured tissue utilizing such sutures. It is contemplated that such procedures and methods may be utilized for human and animal subjects.

Other systems, methods, features and advantages of the embodiments will be, or will become, apparent to one of ordinary skill in the art upon examination of the following figures and detailed description. It is intended that all such additional systems, methods, features and advantages be included within this description and this summary, be within the scope of the embodiments, and be protected by the following claims.

### BRIEF DESCRIPTION OF THE DRAWINGS

The embodiments can be better understood with reference to the following drawings and description. The components in the figures are not necessarily to scale, with emphasis instead being placed upon illustrating the principles of the embodiments. Moreover, in the figures, like reference numerals designate corresponding parts throughout the different views.

FIG. 1 is a schematic view of an anatomical region associated with a knee, in which an implantable biocompatible scaffold has been placed against a damaged ligament, according to an embodiment;

FIG. 2 is a schematic view of an implantable biocompatible scaffold, according to an embodiment;

FIG. 3 is a schematic view of a strand that may comprise part of an implantable biocompatible scaffold, where the strand is formed of a core of non-braided fibers, and an outer layer of braided fibers, according to an embodiment;

FIG. 4 is a schematic view of a machine and process for making a strand comprised of braided and non-braided fibers, according to an embodiment;

FIGS. 5-14 comprise various schematic views depicting different possible configurations of collagen fibers and high strength polymer fibers in a strand, according to various embodiments;

FIG. 15 is a schematic view of a chart showing the tensile properties of a braided strand of the embodiments and a human ACL;

FIG. 16 is a schematic view of a table listing tensile properties of ultra-high-molecular-weight polyethylene fibers and collagen fibers manufactured according to the embodiments;

FIG. 17 is a schematic view of a multi-step process for creating collagen strands, according to an embodiment; and

FIG. 18 is a schematic view of another process for creating collagen strands, according to an embodiment.

### DETAILED DESCRIPTION

The present invention relates generally to novel form factors composed of high strength collagen fibers preferably combined with biocompatible fibers, preferably made of high strength biomaterials. Such biocompatible fibers may be those used in various biotextiles and medical textiles as are known in the art as well as synthetic and semi-synthetic polymers, carbon fibers and steel fibers. Contemplated biocompatible fibers include Polyhydroxy Butyrate (P4HB), Polyvinyl Alcohol (PVA), Reinforcing Cellulose Nanocrystals (CNC), Polycaprolactone (PCL), Polyglycolic acid (PGA), Polygalactin (PG), glycolide-co-e-caprolactone (PGC), Poly-L-lactide (PLLA), Poly-D,L-lactice (PDLLA), Poly-D-Lactide (PDLA), Glycomer 631, PLAGA, PLGA, Polydioxanone (PDO), Cottons, Silk fibroin, Polyethylene (UHMWPE), Polyethylene terephthalate, PEEK, PEKK, Polyester, Polypropylene, Nylon, PTFE, Stainless steel, and Carbon fiber.

One embodiment is directed to braided strands that include a set of high strength collagen fibers braided with a set of high strength polyethylene fibers, preferably high molecular weight polyethylene. Such braided strands have utility for various purposes including, for example, medical uses in orthopedics and surgery.

Some embodiments are directed to implantable biocompatible scaffolds and devices in the form of braided and bundled surgical implants, and surgical and orthopedic devices utilizing such scaffolds, including sutures, as well as related methods for their production and use to support the repair of injured soft and hard tissues, and to stabilize and support various body structures including ligaments, tendons, and joints.

In one embodiment, the scaffold comprises a suture construct that further comprises fibers manufactured using a microfluidic extrusion biomanufacturing process, which is described in further detail below. The suture is designed to be resorbed and replaced with the patient's tissue as the tissue heals completely. The implantable biocompatible scaffold is designed to promote healing in the tissue (such as ligaments, tendons or other suitable tissues) and support accelerated return to activity by enabling early physical therapy compared to conventional alternative treatments.

The sutures and scaffolds of the embodiments may be comprised of one or more braided strands. Each braided strand may be further comprised of fibers that are braided, as well as some non-braided fibers that may be twisted, or otherwise bundled together. In one embodiment, a braided strand is comprised of two types of fibers: high strength collagen fibers and high strength polymer fibers.

As used herein, the term “high strength collagen fiber” refers to a collagen fiber that has an ultimate tensile strength that is substantially greater than that of known manufactured collagen fibers. Preferably, the ultimate tensile strength of collagen fiber embodiments according to the invention have an ultimate tensile strength of at least about 50, 60, 70, 80, 90, 100, 110, 120, 130, 140, 150 or 160 mega Pascals (MPa), where the strength of conventionally manufactured microfibers is about 20 MPa to 40 MPa.

The high strength collagen fibers of the embodiments may be formed according to the processes and composed of the compositions described herein, and further detailed in U.S. Patent Application Publication Number 2020/0246505, published Aug. 6, 2020, and titled “Microfluidic Extrusion,” the entirety of which is herein incorporated by reference, and hereafter referred to as the “Microfluidic Extrusion Application.” Accordingly, the high strength collagen fibers of the embodiments may comprise resorbable micro-fibrous type I bovine fibers that are crosslinked with a benign, biological, and biomimetic crosslinker glyoxal (a crosslinking agent found normally in human ligaments and tendons). The resulting collagen fibers, which are described in further detail below, have a relatively high tensile strength compared to other manufactured collagen strands. In particular, the collagen fibers are sufficiently strong to withstand the mechanical forces applied to the fibers during braiding on high-throughput braiding machines, as are known and used, for example, in the textile and wire industries. See, for example, the various textile and wire braiding systems from manufacturers such as Herzog GmbH (https://herzog-online.com/braidingmachines/) and Seeger USA (https://steegerusa.com/product/medical-braiders/).

The high strength polymer fibers may be high strength polyethylene fibers. As used herein, the term “high strength polyethylene fiber” refers to a fiber with an ultimate tensile strength of at least 80 MPa. In an exemplary embodiment, the high strength polyethylene fibers are ultra-high-molecular-weight polyethylene (hereafter “UHMWPE”) fibers.

Various terms that are used throughout the detailed description and in the claims are collected here for reference.

As used herein, the term “fiber” refers to a filament of material, or to multiple filaments that have been twisted or otherwise bundled together. Fibers may be compared using units that measure the linear density of the fibers. For example, fiber size may be measured using the “tex” unit, which indicates the weight in grams of 1,000 meters of fiber. Dtex, or deci-tex, indicates the weight in grams per 10,000 meters of fiber.

Two or more fibers may be twisted, braided, or otherwise bundled together to form a “strand” of material. Twisted fibers may be twisted around a common axis, or one another, in the same (rotational) direction. By contrast, braided fibers may be interlaced to form more complex patterns. Bundled fibers may be held together by an exterior layer, tie, or other structure.

Parts comprised of two or more fibers that have been braided together may also be referred to as “braided structures.”

The term “over-braiding” refers to the process of braiding two or more fibers over another fiber, collection of fibers, or other suitable structures. A structure that has been formed by an over-braiding process may be referred to as an “over-braided” structure.

As used herein, the term “scaffold” refers to any framework or structure that hold tissues together. A scaffold could comprise a linear structure, such as a suture, a two-dimensional structure, such as a patch or ribbon, or any suitable three-dimensional structure.

The embodiments make reference to several ligaments that may be commonly torn or ruptured and repaired using a scaffold. These include the medial collateral ligament (the “MCL”), the posterior cruciate ligament (the “PCL”), the anterior cruciate ligament (the “ACL”), and the ulnar collateral ligament (the “UCL”). Each of these ligaments are disposed at different anatomical locations at the knee, and may be torn or ruptured during various kinds of physical activities.

The sutures of the present embodiments could be used in a variety of different surgical procedures. In particular, the sutures may be used in surgeries where a ligament must be repaired, internally braced, and/or replaced. Because the sutures of the embodiments have an overall tensile strength that is equal to, or greater than, the tensile strength of some ligaments and tendons in the body, the sutures can be used without other implants, to reinforce and/or repair ligaments such as the ACL, MCL, UCL, and PCL, and tendons such as the supraspinatus in the shoulder, the patellar tendon and Achilles tendon, among others.

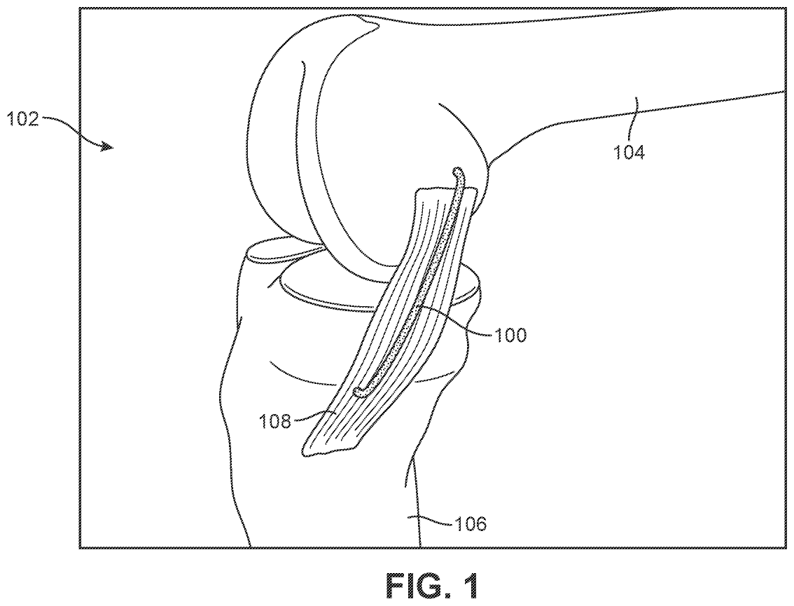

An exemplary procedure that may use a suture to repair a damaged ligament is shown in FIG. 1. Specifically, FIG. 1 is a schematic view of an anatomical region of the leg 102, in which a suture 100 has been attached at its ends to the femur 104 and tibia 106 in order to repair a damaged MCL 108.

As with other structures used for ACL, MCL, and PCL repairs, suture 100 may be implanted using conventional open, minimally invasive and arthroscopic techniques, into the normal ACL, MCL, or PCL anatomical tract using specialized tools, fixation devices and guides that have already been developed and are currently in use by surgeons today.

Once implanted, suture 100 will provide load share and strain relief on the associated ligament. Suture 100 will remodel in vivo into dense regularly oriented connective tissue and exhibit resorption over 6-12 months post implantation.

Although the exemplary embodiment shows the use of a suture comprised of braided strands for use in repairing knee ligaments, it may be appreciated that the embodiments could be used in the repair of tissue in the shoulder, foot, ankle, and other suitable repairs in the body. In some cases, the braided strands of the embodiments could also be used in plastic surgery.

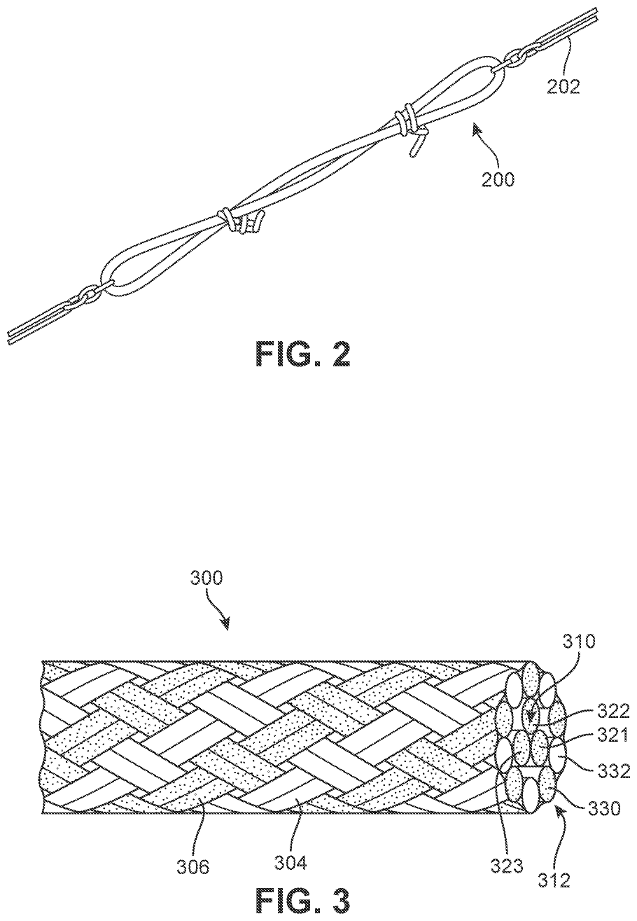

FIG. 2 is a schematic view of a suture 200, shown in isolation. In some cases, suture 200 could comprise a single strand comprised of braided, twisted, or bundled fibers. In other embodiments, however, suture 200 could comprise multiple strands (formed of braided, twisted, or bundled fibers) that have been looped, or otherwise arranged, together. In some cases, a bundle or loop of strands could be joined on each end by non-absorbable polyethylene sutures 202 or other anchors (not shown) for bone fixation.

Sutures of the embodiments could be configured with different geometries. In some embodiments, a suture comprised of one or more braided strands may have a rounded cross-sectional shape. Other embodiments of sutures could have a flattened geometry. Still other embodiments of sutures could include combinations of flattened and rounded portions. For example, one embodiment of a suture could comprise a flattened middle portion with rounded ends that facilitate tie down properties for the suture. Likewise, at the level of an individual strand, braided strands could also be configured with flattened geometries, rounded geometries, or a combination of flattened and rounded geometries. As described below, one way to achieve a rounded strand geometry is to over-braid fibers onto a core of straight or twisted fibers. Flattened braided strands can be made using flat-braiding techniques.

While the embodiment of FIG. 2 depicts a suture comprised of braided strands, other embodiments may comprise fibers braided strands incorporated into a variety of other suitable geometries and structures, including, for example, patches, braces, and tapes.

FIG. 3 is a schematic view of a section of a single strand 300. Strand 300 may be further comprised of a set of collagen fibers 304 and a set of polymer fibers 306. Sets of fibers may include one, two, three, or more than three fibers. For purposes of illustration, polymer fibers are shown with shading in the Figures to distinguish them from the collagen fibers.

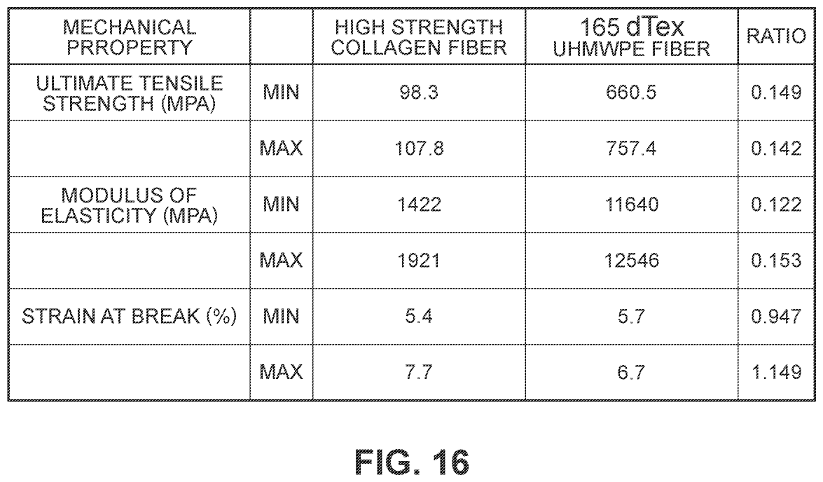

Collagen fibers 304 may be high strength collagen fibers according to the embodiments. The high strength collagen fibers may have sufficiently higher tensile strength than conventionally manufactured collagen fibers. Specifically, the fibers may be strong enough to withstand stresses imparted to the fibers when manipulated on industrial scale braiding machines. The specific tensile properties of these high strength collagen fibers are described in further detail below and shown, for example, in FIG. 16.

Polymer fibers 306 may comprise a high strength polyethylene material. More specifically, in some embodiments, polymer fibers 306 are ultra-high-molecular-weight polyethylene fibers 306.

The fibers of strand 300 may be further arranged into a core 310 and an outer layer 312. Core 310 may comprise a plurality of fibers with a straight or twisted configuration. That is, the fibers in core 310 may not be braided. In contrast, outer layer 312 is comprised of fibers that have been over-braided along the fibers of core 310. Over-braiding fibers onto a core of straight or twisted fibers may help strand 300 take on a generally rounded cross-sectional shape.

In the exemplary embodiment, core 310 includes three fibers. These include a first polymer fiber 321, a second polymer fiber 322, and a third polymer fiber 323. By contrast, outer layer 312 is seen to include eight fibers. These include four polymer fibers 330 that alternate with four collagen fibers 332 along the exterior of strand 300.

For purposes of illustration, strand 300 is shown with a particular braided pattern, visible along its side. However, it may be appreciated that embodiments are not limited to a particular braiding pattern. Any suitable braided pattern could be used and selected according to various factors such as the number of fibers used and the sizes of the fibers.

Scaffolds, including sutures, of the present embodiments can be formed from a single strand, or from multiple strands that are looped, bundled, braided, twisted, or otherwise joined together. For example, referring back to FIG. 2, suture 200 may be formed from a single strand, such as strand 300 shown in FIG. 3, or else from multiple strands like strand 300 that have been bundled, twisted, and/or braided together into the looped configuration shown in FIG. 2.

In still other embodiments, strands similar to strand 300 could be arranged into two-dimensional configurations to form ribbons, rectangular patches, or other two-dimensional implants used for tissue repair, tissue augmentation, wound closure and delivery of biological agents such as cells, cell-based products, genes, growth factors, small molecules, drugs or other therapeutic agents as would be known to persons skilled in the art.

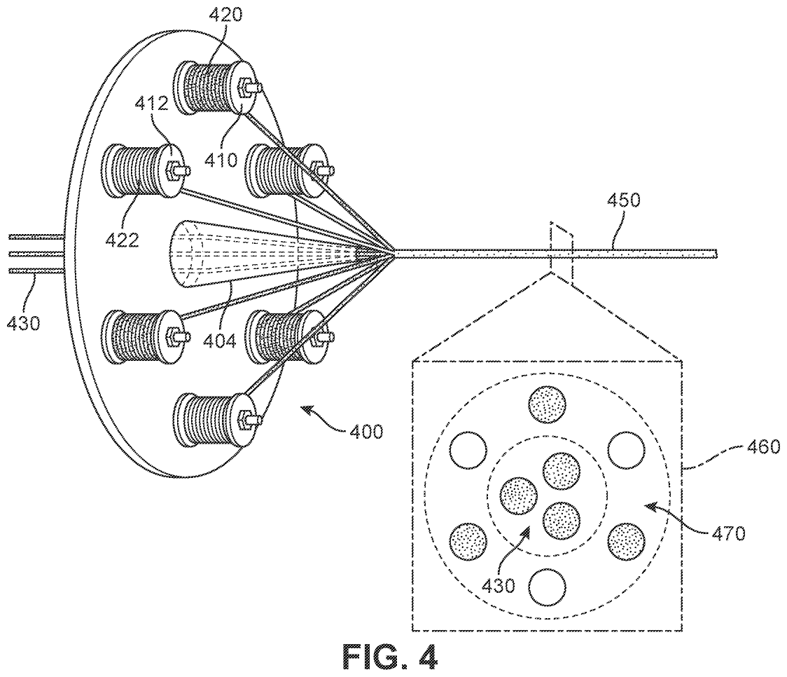

FIG. 4 is a schematic view of an exemplary braiding process for forming a strand that comprises a core of fibers and an over-braided outer layer of fibers. Referring to FIG. 4, a braiding machine 400 may be used to over-braid fibers onto a central core, thereby forming a braided strand 450. Braiding machines may generally include spools, or bobbins, that are moved or passed along various paths on the machine by carriers.

For purposes of illustration, braiding machine 400 is shown with six bobbins that ride on six carriers (not shown). However, it may be appreciated that in other embodiments additional bobbins/carriers could also be used. In one embodiment, for example, a 24-carrier braiding machine could be used.

Each carrier includes a bobbin with either high strength polymer fiber or high strength collagen fiber. For example, a first bobbin 410 holds a high strength polymer fiber 420. Likewise, a second bobbin 412 holds a high strength collagen fiber 422. As the braiding machine is run and bobbins are passed around between carriers, braided strands extending from the bobbins towards a center of the machine may converge at a “braiding point”.

Core fibers 430 are fed from behind into a central channel of machine 400 and exit out through a nozzle 404. Strands from each carrier are drawn out to a braiding point just beyond nozzle 404 so that the strands can be over-braided around core fibers 430 to form a two layered construction.

An enlarged cross-sectional view 460, taken along a section of braided strand 450, shows six fibers braided together in an outer layer 470. The outer layer 470 of fibers encircle the core fibers 430. In this exemplary embodiment, core fibers 430 all comprise polymer fibers. Also, outer layer 470 is comprised of three collagen fibers and three polymer fibers arranged in an alternating configuration. For reference, dotted lines are shown in the enlarged cross-sectional view of braided strand 450 to indicate approximate boundaries of the core and outer layer. However, these boundaries are not intended to represent physical structures or barriers.

A braided strand can be configured with different properties according to the number, type, and spatial arrangement of fibers used, and the types of copolymer(s) used in the fibrous constructs. These different properties include, but are not limited to: tensile strength, elasticity, size (e.g., diameter), weight, biocompatibility, visibility, and cost.

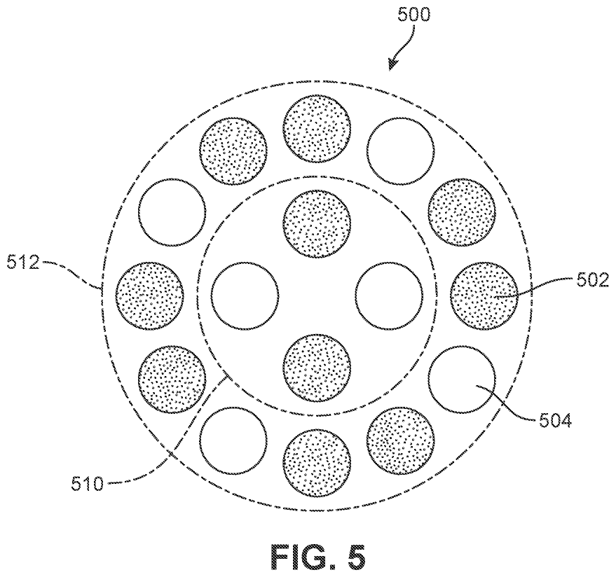

FIGS. 5-14 are schematic views of various possible fiber configurations within a braided strand. As already described, the embodiments include an inner core of fibers and an over-braided outer layer of fibers. It may be appreciated that the material properties of a braided strand may depend on the number, size, shape, type, and spatial arrangements of fibers both within the core and in the outer over-braided layer.

FIG. 5 is a schematic view of an exemplary braided configuration. In this embodiment, a braided strand 500 includes sixteen total fibers, including both high strength polymer fibers 502 (specifically UHMWPE fibers) and high strength collagen fibers 504. More specifically, core 510 comprises four fibers, while outer layer 512 is comprised of the remaining twelve fibers. In this example, core 510 includes two polymer fibers and two collagen fibers. Of the remaining fibers in outer layer 512, four are collagen fibers while eight are polymer fibers. More specifically, the fibers in outer layer 512 are arranged at this particular location along the strand such that there are two polymer fibers disposed between each pair of adjacent collagen fibers. The relatively large number of polymer fibers results in a strand with about ninety percent of the tensile strength of a similarly configured strand in which each of the sixteen fibers are UHMWPE polymer fibers (that is, in a similarly constructed strand where all fibers are polymer fibers).

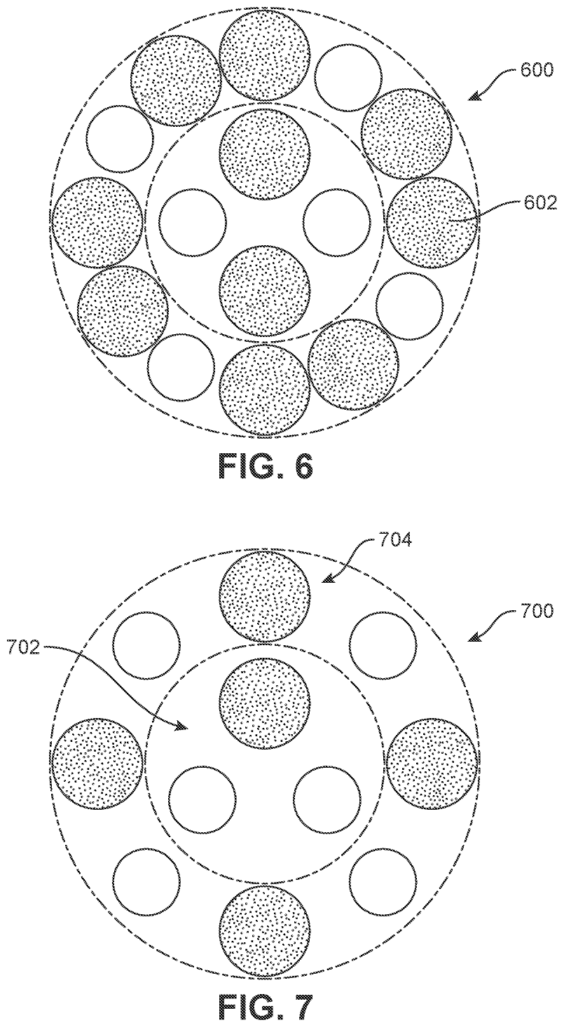

FIG. 6 is a schematic view of another exemplary braided configuration. The configuration of braided strand 600 in FIG. 6 may be substantially similar to that of braided strand 500 in FIG. 5. However, the polymer fibers 602 in this embodiment have a larger linear density than the polymer fibers 502 of the previous embodiment. For purposes of illustration, this increased linear density of polymer fibers 602 compared to polymer fibers 502 is represented with larger diameter fibers. In some embodiments the polymer fibers of braided strand 500 have a linear density of 110 dTex, while the polymer fibers of braided strand 600 have a linear density of 165 dTex. This increased size for the polymer fibers may provide better cushioning for the adjacent collagen fibers.

FIG. 7 is a schematic view of another exemplary braided configuration. The configuration of FIG. 7 includes a braided strand 700 with three core fibers 702 and eight fibers 704 in the outer layer (for a total of eleven fibers). The three core fibers are further comprised of two collagen fibers and a single polymer fiber. The outer layer includes four polymer fibers alternating with four collagen fibers. This configuration provides a strand with a higher collagen percentage relative to the previous embodiments (approximately 55% for strand 700 vs. approximately 38% for strand 500 and strand 600).

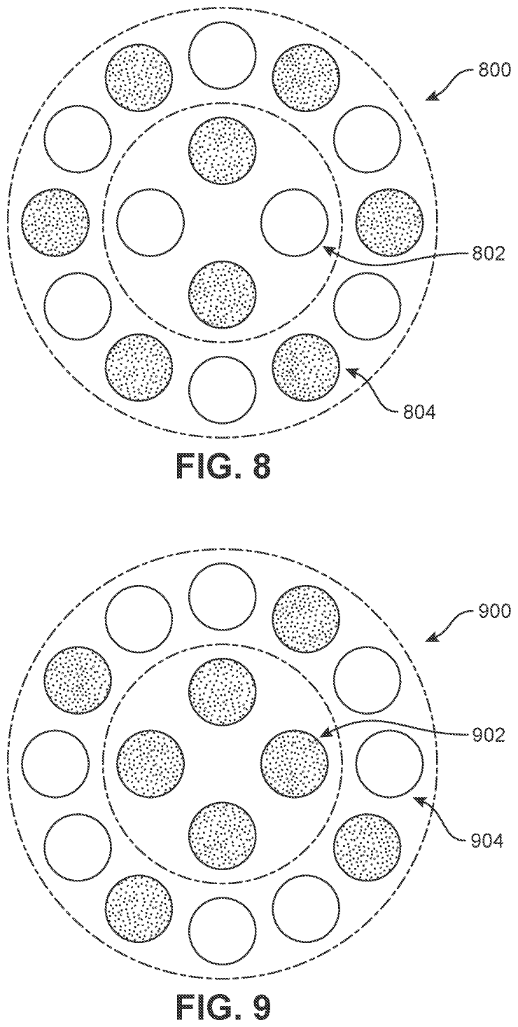

FIG. 8 is a schematic view of another exemplary braided configuration. The configuration of FIG. 8 includes a braided strand 800 with four core fibers 802 and twelve outer fibers 804 in the outer layer (for a total of sixteen fibers). Both the core and outer layer have an equal number of collagen and polymer fibers, so that overall the strand has eight collagen fibers and eight polymer fibers. Braided strand 800 retains approximately 85% of the tensile strength of a similar strand comprised solely of polymer fibers, and is comprised of approximately 50% collagen.

FIG. 9 is a schematic view of another exemplary braided configuration. The configuration of FIG. 9 includes a braided strand 900 with four core fibers 902 and twelve outer fibers 904 in the outer layer (for a total of sixteen fibers). In this example, the core is comprised only of polymer fibers. Of the remaining fibers in the outer layer, eight are collagen fibers while four are polymer fibers. More specifically, the fibers in the outer layer are arranged at this particular section of the braided strand such that there are two collagen fibers disposed between each pair of adjacent polymer fibers. The relatively large number of polymer fibers results in a strand with about ninety percent of the tensile strength of a similarly configured strand in which each of the sixteen fibers are UHMWPE polymer fibers (that is, in a strand that lacks any collagen fibers). Moreover, this embodiment positions all of the collagen strands on the outside of the strand, where they can more easily contact tissue in the body to better facilitate healing.

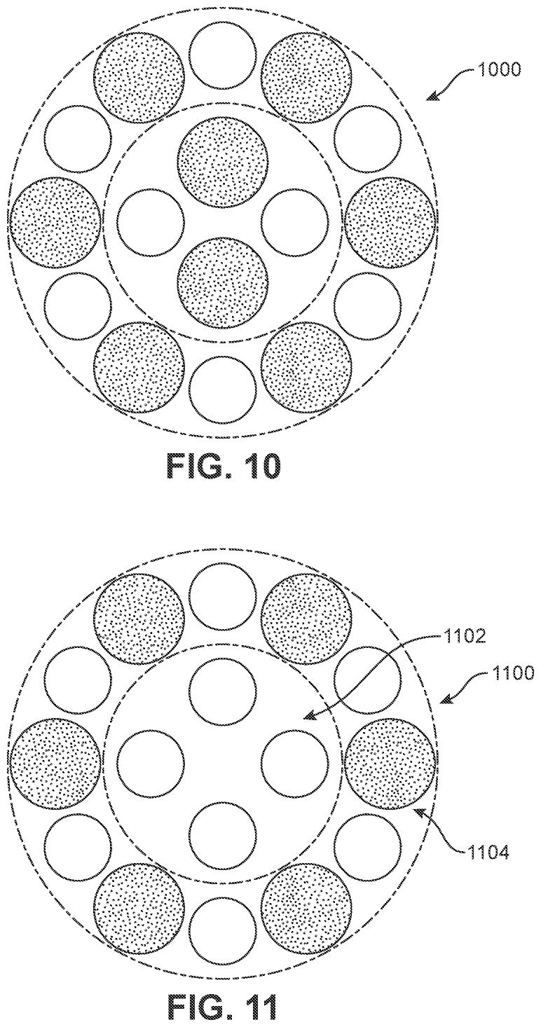

FIG. 10 is a schematic view of another exemplary braided configuration. The configuration of a braided strand 1000 in FIG. 10 may be substantially similar to that of braided strand 900 in FIG. 9. However, whereas the polymer fibers of braided strand 900 have a linear density of 110 dTex, the polymer fibers of braided strand 1000 have a linear density of 165 dTex. This increased size for the polymer fibers may facilitate improved cushioning for the collage fibers on the exterior of the strand.

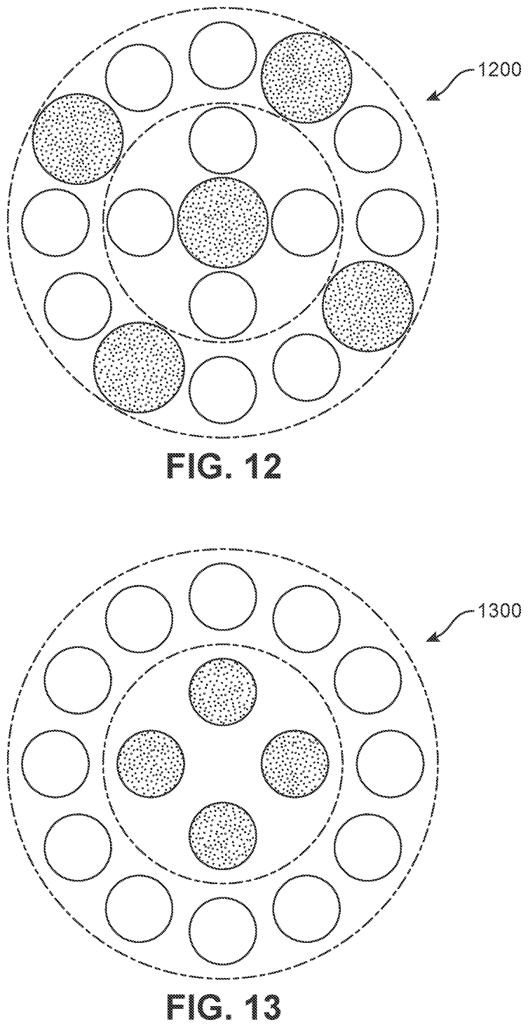

FIGS. 11-12 illustrate schematic views of braided configurations where more than half the fibers in each braided strand are collagen fibers. Specifically, FIG. 11 depicts a braided strand 1100 with six polymer fibers and ten collagen fibers. In this case, the core 1102 is comprised of four collagen fibers, while the outer layer 1104 include six collagen fibers alternating with six polymer fibers.

In FIG. 12, braided strand 1200 is comprised of five polymer fibers and twelve collagen fibers. Moreover, the core includes four collagen fibers surrounding a polymer fiber (for a total of 5 core fibers). The outer layer comprises eight collagen fibers and four polymer fibers. Braided strand 1200 retains approximately 75% of the tensile strength of a similar strand comprised solely of polymer fibers.

FIG. 13 is an exemplary embodiment of a strand 1300 comprised of a core consisting of only polymer fibers and an outer layer comprised of only collagen fibers braided together. Here, the core polymer fibers help provide a rounded shape for the strand and add tensile strength to the strand. But by using all collagen fibers along the outside, the strand can promote healing everywhere that the exterior of the strand comes into contact with damaged tissue.

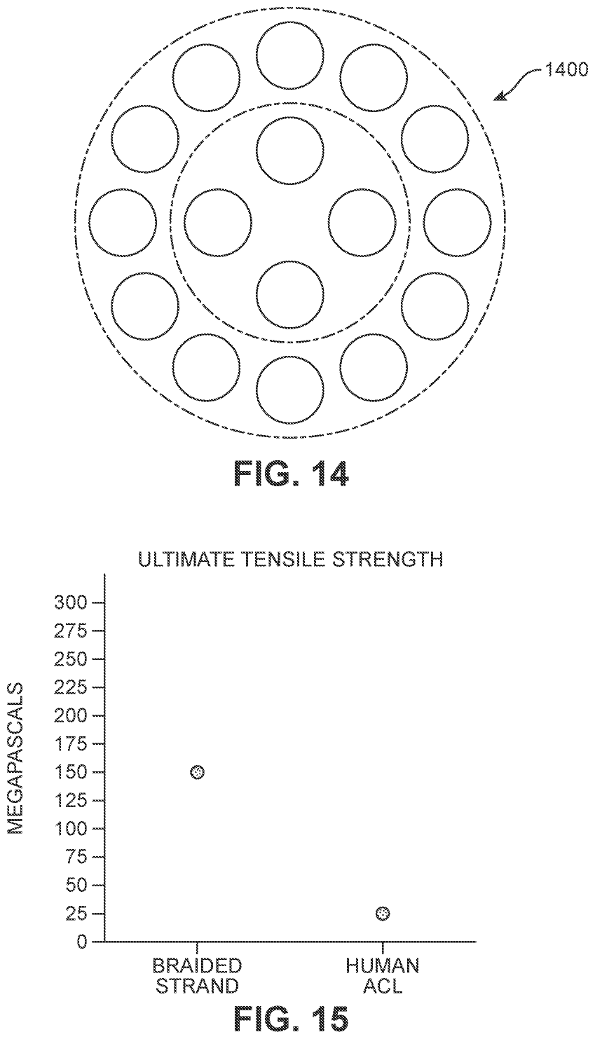

FIG. 14 is an exemplary embodiment of a strand 1400 consisting only of collagen fibers. In this case both the core and outer braided layer are comprised of only collagen strands. Using only collagen may maximize the potential of the strand to contribute to healing, by eliminating the presence of polymer fibers which may be non-bioabsorbable and which may not promote new tissue growth. Moreover, the use of high strength collagen strands as disclosed in the embodiments may provide a strand that has similar, or greater, ultimate tensile strength than an associated ligament or other tissue to be repaired using the strand.

As seen in FIGS. 5-14, various configurations of a braided strand may include one or more collagen strands on in the outer layer. This not only promotes healing, but also facilitates better tie down properties for sutures comprised of the braided strand, since the collagen strands are generally “stickier” than the UHMWPE strands. By providing configurations of braided strands where a significant fraction of the strands in the outer layer are collagen strands (e.g., more than 30% of the total strands), the embodiments eliminate any need for introducing another type of strand and/or coating for strands that might be required to ensure sutures comprised of the braided strands can be tied down.

The embodiments comprise strands formed by braiding high strength polyethylene fibers with collagen fibers having a relatively high tensile strength. For purposes of describing the tensile properties of various fibers, the embodiments make use of various terminology including ultimate tensile strength, yield strength, modulus of elasticity, and strain at break. As used herein, “ultimate tensile strength,” or UTS, is the maximum stress that a material can withstand while being stretched or pulled before breaking. As used herein, the “yield strength” or “yield stress” is the stress corresponding to the yield point at which a material begins to deform plastically. As used herein, the “modulus of elasticity” is a measure of the stiffness of an elastic material. Specifically, it is a ratio of stress along an axis to strain along the same axis. As used herein, the term “strain at break” is a measure of the change in length of a material at the point where the material breaks under tension.

The braided strands of the embodiments have sufficiently greater tensile strength than corresponding ligaments and tendons in the body, as seen in FIG. 15, which compares the tensile strength of an exemplary braided strand according to the embodiments with the tensile strength of a human ACL. In this example, an exemplary braided strand has an ultimate tensile strength of approximately 150 MPa, while a human ACL has a UTS between 25 and 50 MPa.

To achieve braided strands of the embodiments, collagen fibers with relatively high tensile strengths are used, as already discussed. FIG. 16 is a schematic table showing various tensile properties for high strength collagen fibers according to the embodiments and of UHMWPE fibers manufactured according to known processes. In particular, the values shown in the chart are for a high strength collagen fiber that is comprised of individual collagen filaments that have been bundled together to form a single continuous fiber with an average diameter approximately in a range between 90 μm and 180 μm. The UHMWPE strands are 165 dTex strands with an average diameter approximately in a range between 210 μm and 410 μm. The values of the tensile properties for the high strength collagen and UHMWPE strands disclosed here were determined using standard techniques for measuring ultimate tensile strength and other tensile properties. The fibers were tested in similar conditions.

The Table in FIG. 16 shows minimum and maximum values for ultimate tensile strength, modulus of elasticity, and strain at break for the two listed fibers. Additionally, a third column shows the ratios of the values for the high strength collagen to the values for the UHMWPE.

As seen in the table of FIG. 16, for the particular samples tested, the ultimate tensile strength (UTS) of the high strength collagen fibers varies approximately between 98 and 110 Megapascals (MPa), whereas the UHMWPE fibers have a UTS that varies approximately between 660 MPa and 760 MPa. Therefore, the UTS of the high strength collagen fibers varies approximately in a range between 14% and 15% of the UTS of the UHMWPE fibers.

In FIG. 16, comparison values for the modulus of elasticity and strain at peak are also given. These show that for the particular samples tested, the high strength collagen fibers have a modulus of elasticity that is approximately in a range between 12% and 15% of the modulus of elasticity of the UHMWPE fibers. Similarly, the high strength collagen fibers have a strain at break that is approximately in a range between 90% to 115% of the strain at break of the UHMWPE fibers.

The mechanical properties of the high strength collagen fibers described herein allow for the manipulation of the fibers and their production into the braided strands of the embodiments. While UHMWPE is stronger than these high strength collagen strands, the collagen strands are still strong enough to withstand the stresses imparted to the strands by high throughput and conventional braiding machines that allow the braided strands to be manufactured at scale. Moreover, the relatively high strength of the collagen fibers allows for greater flexibility in constructing a braided strand using both collagen and UHMWPE fibers. Because the high strength collagen fibers provide some strength to the braided strand, fewer UHMWPE fibers may be required to maintain a minimum desired tensile strength and other parameters for the braided strand. This allows for a greater ratio of collagen fibers to UHMWPE fibers, which is more suitable for healing damaged tissue.

In still other embodiments, collagen fibers can be manufactured with a range of different tensile strengths, by varying the cross-linking compounds used, collagen collection methods, and other suitable characteristics. It may be appreciated, therefore that the values given for various mechanical properties of high strength collagen fibers are only intended as examples and should not be construed as limiting.

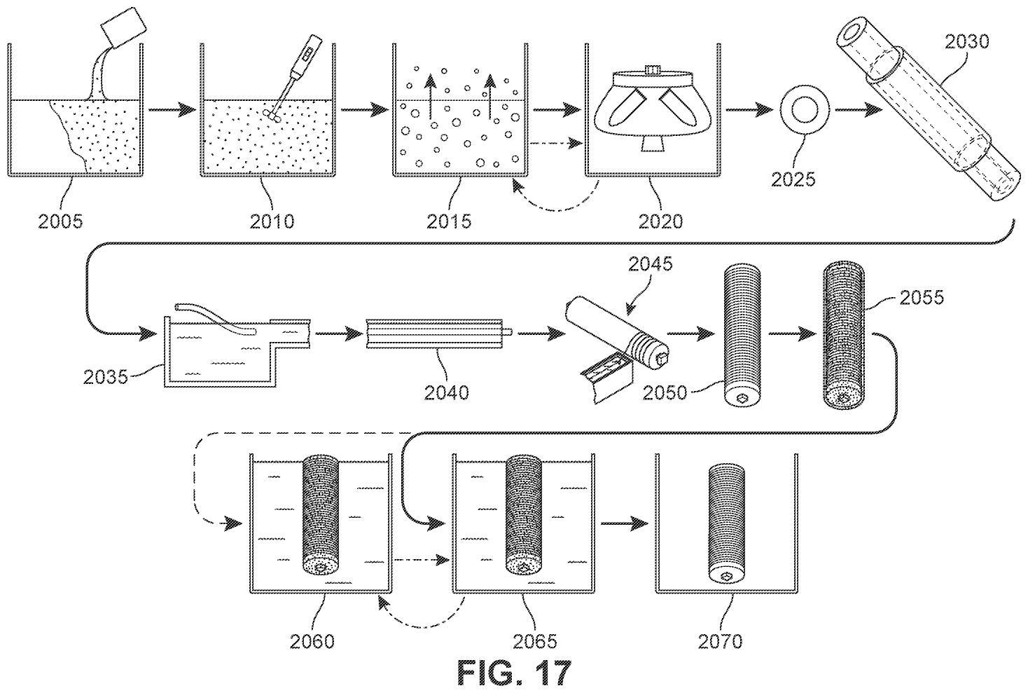

FIG. 17 illustrates a schematic multi-step process, and associated system, whereby high strength collagen fibers can be formed. That is, collagen fibers that have substantially greater tensile properties than conventionally manufactured collagen fibers. The system and method may be described as comprising four sections or manufacturing areas. A collagen solution is prepared in the first section, and collagen fiber is formed in the second section. The collagen fiber then is collected in the third section and then may be post-processed to yield wet or dry collagen fiber in the fourth section, post-treatments or end of treatment.

The steps in the system and method illustrated in FIG. 17 may be grouped into four categories, as follows: (1) Preparing Collagen Solution, including step 2005 through step 2020; (2) Forming Collagen Fiber, including step 2025 through step 2030; (3) Collecting Collagen Fiber, including step 2035 through step 2050; and (4) Post-Treatment or End Treatment, including step 2055 through step 2080.

As seen at step 2005 of FIG. 17, collagen is combined with an acidic solution and stirred thoroughly at step 2010. In some embodiments, the acid is between about 0.01 M and about 0.50 M acetic acid. In other embodiments, the acid is between about 0.01 M and about 0.50 M hydrochloric acid. The solution may be degassed at step 2015, and then centrifuged at step 2020 to remove residual bubbles. Resultant collagen solution is extruded from a needle, and there may be a second needle co-axial therewith that supplies a formation buffer solution in step 2025. The resultant forming fiber may continue in through a formation tube in step 2030. The resultant product is a formed collagen fiber.

The fiber then continues to a collection system, wherein the fiber is separated from the formation buffer solution at step 2035 and dehydrated at step 2040. The collagen fiber is recovered at step 2045 and air-dried at step 2050. Then, postprocessing may be carried out, as illustrated at step 2055, step 2060, step 2065, and step 2070. Air-dried collagen fiber on a spool is submerged in cross-linking solution at step 2055, optionally washed at step 2060, air-dried at step 2065, and desiccated to form dried fiber at step 2070. As illustrated in FIG. 17 by the dot-dash line, material may be optionally washed at step 2060, dried at step 2065, and returned to wash step 2060.

Alternatively, collagen is injected into a bath of formation solution to form a fiber. In this system, a second needle for coaxial injection of formation buffer is not necessary. Collagen thus injected is introduced to a collection system through dehydration at step 2040. The fiber then is processed in accordance with the remainder of the processing steps.

FIG. 17 provides a generalized view of a system and method for carrying out an embodiment of the disclosure. Additional details and disclosure are included in the Microfluidics Application.

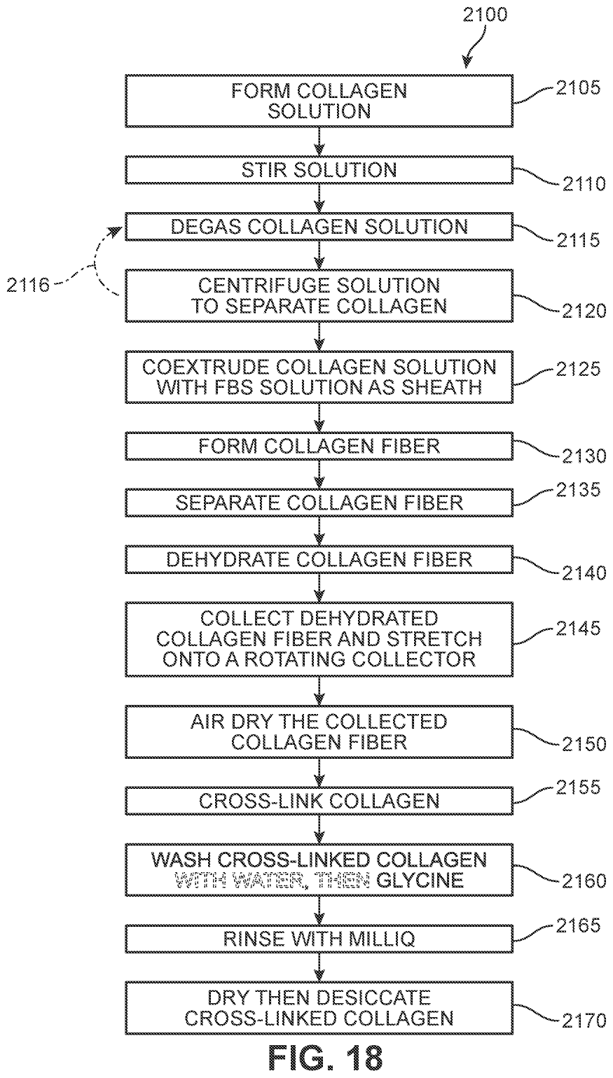

Another embodiment of the exemplary method is shown in FIG. 18. Method 2100 begins with step 2105, where a collagen solution is formed. A biopolymer may be mixed with the collagen. Collagen is dissolved in an acidic solution to form a viscous solution. The solution is stirred at step 2110 to ensure thorough mixing. The mixed solution may have entrapped gas, and so may be degassed one or more times in degasser step 2115. The collagen solution then may be centrifuged in step 2120. Optionally, the degas/centrifuge steps may be repeated, as shown by the dot-dash line 1316 on FIG. 18, to reduce the volume of gas entrapped in the solution. Thus-prepared collagen solution is formed into a collagen fiber by coaxial extrusion with a formation buffer solution that serves as a sheath for the fiber core in step 2125. The formation buffer solution volumetric flow rate typically is at least twice the volumetric flow rate of the forming collagen. This arrangement suppresses formation of individual fibrils; stretches and orients the fiber; and may smooth the surface of the fiber by imparting flow-induced crystallization to the fiber.

The collagen fiber then is collected. As formation of the collagen fiber is completed at step 2130, the collagen then is separated from the formation buffer solution at step 2135 and dehydrated in a dehydrating solution at step 2140.

The dehydrated collagen then is collected on a rotating spool in step 2145, which further stretches the fiber by rotating at a rate greater than, and typically about twice, the rate at which the fiber is supplied from dehydrating solution step 2140. Thus, collected fiber then is air-dried on the spool in step 2150.

In an alternative embodiment, collagen solution is formed into a collagen fiber by direct injection into formation buffer solution. Thus, step 2125 is skipped. The fiber is collected, separated from formation buffer solution, and dehydrated in a dehydrating solution at step 2140. The fiber is collected on a rotating spool in step 2145, which collects fiber at a speed of between about 2 times the formation speed and about 4 times the formation speed.

Fiber that has been air-dried on the spool then may be postprocessed. Fiber may be cross-linked in a cross-linking solution at step 2155, and then may be rinsed at step 2160. The fiber then is air dried at step 2165 and desiccated at step 2170 to yield dry cross-linked collagen fiber.

The equipment used in making collagen fiber is made of conventional materials of construction suitable for resisting attack by any of the raw materials used to make collagen fiber in accordance with embodiments of the disclosure. Metals, plastics, and other materials have properties and characteristics suitable to resist attack by raw materials, intermediates, solvents, and products during manufacture of collagen fiber.

The process described herein is used to form a collagen fiber that has substantially better tensile properties than other collagen fibers that may be available commercially. Specifically, the collagen fibers may have one or more of the following characteristics: (1) an ultimate tensile strength of at least 80 MPa; (2) a modulus of elasticity of at least 1200 MPa; (3) a strain at break of between about 4 percent and about 12 percent elongation; and (4) an average fiber diameter between about 90 μm and about 180 μm. Moreover, the collagen fiber at least maintains its strength after soaking in biological fluid for about 1 hour.

Still further, the fiber exhibits an ordered, longitudinally-oriented structure, and the fiber allows infiltration of cellular growth.

As described above, the disclosed scaffold or suture construct is intended to be implanted into or along the normal anatomical tract of various ligaments or other suitable tissues, to aid in the repair of type 1 and 2 ruptures/tears. Performance testing has demonstrated that the device will have the required mechanical and physical properties to function as a useful construct in ACL and PCL surgical repairs with the product having average yield loads exceeding those of native ligaments. Preferably, such devices will be terminally sterilized using electron beam sterilization and will be intended for single use only.

The device will provide load share and strain relief on the primary ACL, MCL, or PCL surgical repair. The device will remodel in vivo into dense regularly oriented connective tissue and exhibit resorption over 6-12 months post implantation. As with other suture constructs used for ACL and PCL repairs, the device will be implanted, using conventional arthroscopic techniques, into the normal ACL or PCL anatomical tract using specialized tools, fixation devices and guides that have already been developed and are currently in use by surgeons today.

While various embodiments have been described, the description is intended to be exemplary, rather than limiting and it will be apparent to those of ordinary skill in the art that many more embodiments and implementations are possible that are within the scope of the embodiments. Although many possible combinations of features are shown in the accompanying figures and discussed in this detailed description, many other combinations of the disclosed features are possible. Any feature of any embodiment may be used in combination with or substituted for any other feature or element in any other embodiment unless specifically restricted. Therefore, it will be understood that any of the features shown and/or discussed in the present disclosure may be implemented together in any suitable combination. Accordingly, the embodiments are not to be restricted except in light of the attached claims and their equivalents. Also, various modifications and changes may be made within the scope of the attached claims.

What is claimed is:

1. An implantable biopolymer scaffold, the scaffold including at least one braided strand, the braided strand consisting essentially of high strength collagen fibers and high strength biocompatible fibers.

2. The implantable biopolymer scaffold according to claim 1, wherein the biocompatible fibers are selected from the group consisting of high strength polyethylene fibers and ultra-high-molecular-weight polyethylene fibers.

3. The implantable biopolymer scaffold according to claim 1, wherein the scaffold is selected from the group of form factors consisting of a brace, patch, tape and a suture, such as round suture, flat suture and round-flat-round suture.

4. The implantable biopolymer scaffold according to claim 1, wherein at least some of the high strength collagen fibers are braided with at least some of the high strength polyethylene fibers.

5. The implantable biopolymer scaffold according to claim 1, wherein the braided strand comprises a central core and an outer layer over-braided around the central core.

6. The implantable biopolymer scaffold according to claim 5, wherein the central core comprises unbraided fibers.

7. The implantable biopolymer scaffold according to claim 6, wherein the outer layer comprises high strength collagen fibers braided with high strength polyethylene fibers.

8. The implantable biopolymer scaffold according to claim 1, wherein at least half of the fibers comprising the braided strand are collagen fibers.

9. The implantable biopolymer scaffold according to claim 1, wherein the high strength polyethylene fibers have substantially larger diameters than the high strength collagen fibers.

10. An implantable biopolymer scaffold, the scaffold comprising:
a set of high strength collagen fibers braided with a set of high strength polyethylene fibers;
wherein a high strength collagen fiber of the set of high strength collagen fibers has a first ultimate tensile strength, and wherein a high strength polyethylene fiber of the set of high strength polyethylene fibers has a second ultimate tensile strength; and
wherein the first ultimate tensile strength is at least one percent of the second ultimate tensile strength.

11. The implantable biopolymer scaffold according to claim 10, wherein the first ultimate tensile strength is selected from the group consisting of at least about 1 percent, 3 percent, at least 5 percent, at least about 10 percent, at least about 15 percent and at least about 20 percent of the second ultimate tensile strength.

12. The implantable biopolymer scaffold according to claim 10, wherein the first ultimate tensile strength is at least about ten percent of the second ultimate tensile strength.

13. The implantable biopolymer scaffold according to claim 10, wherein the first ultimate tensile strength has a value of at least about 50, 60, 70, 80, 90, 100, 100, 120, 130, 140, 150 or 160 MPa.

14. The implantable biopolymer scaffold according to claim 10, wherein the first ultimate tensile strength has a value of at least 100 MPa.

15. A braided strand comprising a plurality of high strength collagen fibers and high strength polyethylene fibers.

16. A braided strand comprising:
a set of high strength collagen fibers braided with a set of high strength polyethylene fibers;
wherein a high strength collagen fiber of the set of high strength collagen fibers has a first ultimate tensile strength, and wherein a high strength polyethylene fiber of the set of high strength polyethylene fibers has a second ultimate tensile strength; and
wherein the first ultimate tensile strength is at least about one percent of the second ultimate tensile strength.

17. The braided strand according to claim 16, wherein the high strength polyethylene fibers are ultra-high-molecular-weight polyethylene fibers.

18. The braided strand according to claim 16, wherein the first ultimate tensile strength is selected from the group consisting of at least about 1 percent, at least about 3 percent, at least about 5 percent, at least about 10 percent, at least about 15 percent and at least about 20 percent of the second ultimate tensile strength.

19. The braided strand according to claim 16, wherein the first ultimate tensile strength has a value of at least about 50, 60, 70, 80, 90, 100, 100, 120, 130, 140, 150 or 160 MPa.

20. A method of repairing an injured joint, ligament or tendon, comprising the step of implanting an implantable biopolymer scaffold according to claim 1.

21. A method of securing a medical implant in the desired position, comprising the step of fastening the implant with a suture according to claim 3.

22. A method of closing an incision or wound or repairing injured tissue comprising the step of suturing the incision, wound or tissue with a suture according to claim 3.
